(19) United States
(12) Patent Application Publication
(10) Pub. No.: US 2018/0299658 A1
(43) Pub. Date: Oct. 18, 2018

(54) SYSTEMS AND METHODS OF OPTICAL COHERENCE TOMOGRAPHY STEREOSCOPIC IMAGING FOR IMPROVED MICROSURGERY VISUALIZATION

(71) Applicant: Duke University, Durham, NC (US)

(72) Inventors: Oscar M. Carrasco-Zevallos, Durham, NC (US); Brenton Keller, Durham, NC (US); Liangbo Shen, Durham, NC (US); Christian B. Viehland, Durham, NC (US); Cynthia A. Toth, Durham, NC (US); Joseph A. Izatt, Durham, NC (US)

(21) Appl. No.: 15/568,198

(22) PCT Filed: Apr. 22, 2016
(86) PCT No.: PCT/US16/28862

§ 371 Date: Oct. 20, 2017

Related U.S. Application Data
(60) Provisional application No. 62/151,526, filed on Apr. 23, 2015.

(51) Int. Cl. G02B 21/36; G02B 21/22; A61B 3/10; G02B 21/00; A61B 3/13; H04N 13/398; H04N 13/106

(52) U.S. Cl. CPC G02B 21/367; G02B 21/22; A61B 3/102; G02B 21/0012; G02B 21/368; A61B 3/13; G06T 5/50; H04N 13/106; G06T 2207/10012; G06T 2207/20192; G06T 2207/30041; G06T 2207/10101; G06T 2207/20024; H04N 13/398

(57) ABSTRACT

Systems and methods of optical coherence tomography stereoscopic imaging for microsurgery visualization are disclosed. In accordance with an aspect, a method includes capturing a plurality of cross-sectional images of a subject. The method includes generating a stereoscopic left image and right image of the subject based on the cross-sectional images. Further, the method includes displaying the stereoscopic left image and the right image in a display of a microscope system.

### CROSS REFERENCE TO RELATED APPLICATION

This application claims the benefit of U.S. Provisional Patent Application No. 62/151,526, filed Apr. 23, 2015 and titled SYSTEMS AND METHODS FOR REAL-TIME OPTICAL COHERENCE TOMOGRAPHY TO ENHANCE VISUALIZATION OF MICROSURGERY, the disclosure of which is incorporated herein by reference in its entirety.

### FEDERALLY SPONSORED RESEARCH OR DEVELOPMENT

The technology disclosed herein was made in part with government support under Federal Grant No. EY023039 awarded by the National Institutes of Health (NIH). The United States government has certain rights in the technology.

### TECHNICAL FIELD

The present subject matter relates to medical imaging. More particularly, the present subject matter relates to systems and methods of optical coherence tomography stereoscopic imaging for microsurgery visualization.

### BACKGROUND

Ophthalmic surgery is typically performed with a stereoscopic surgical microscope that provides a wide field en face view of the surgical field and limited depth perception. Surgeons often rely on indirect cues for depth information, which may be insufficient for precise depth localization of tissue-tool interfaces. Many ophthalmic surgical procedures, such as corneal dissections and external limiting membrane peeling, necessitate precise axial manipulation of tissue. Therefore, direct three-dimensional (3D) visualization of dynamic surgical maneuvers can be very useful in ophthalmic surgery.

Optical coherence tomography (OCT) enables micron-scale tomographic imaging of posterior and anterior segments of the human eye and can provide direct axial visualization of ophthalmic surgery. While portable and hand-held OCT systems have been previously implemented for intraoperative imaging, these systems require displacement of the surgical microscope and thus necessitate pauses in surgery for imaging. To eliminate this necessity, microscope integrated OCT (MIOCT) systems have been developed for concurrent imaging with OCT and the surgical microscope. In such MIOCT systems, which are coaxial with the surgical microscope, live recording of surgical maneuvers are enabled.

There is a continuing need for improved systems and techniques for improving the display of images of the surgical field to surgeons and other healthcare professionals. Particularly, it is desired to provide improvements in the display and manipulation of images during ophthalmic surgery.

### SUMMARY

Disclosed herein are systems and methods of optical coherence tomography stereoscopic imaging for microsurgery visualization. In accordance with an aspect, a method includes capturing a plurality of cross-sectional images of a subject. The method includes generating a stereoscopic left image and right image of the subject based on the cross-sectional images. Further, the method includes displaying the stereoscopic left image and the right image in a display of a microscope system.

### BRIEF DESCRIPTION OF THE DRAWINGS

The foregoing aspects and other features of the present subject matter are explained in the following description, taken in connection with the accompanying drawings, wherein:

FIG. 1 is a schematic diagram of an example 4D MIOCT system in accordance with embodiments of the present disclosure;

FIG. 2 illustrates a graph of a fall off plot showing data acquired by the system 100 shown in FIG. 1;

FIG. 3 illustrates an image of an example microscope system including an MIOCT scanner and heads-up display (HUD) in accordance with embodiments of the present disclosure;

FIG. 4 is an image showing an MIOCT volume generated in accordance with embodiments of the present disclosure

FIG. 5 is an image of a B-scan acquired in accordance with embodiments of the present disclosure;

FIGS. 6A and 6B are images of a left ocular view and a second ocular view respectively after projection of MIOCT data;

FIGS. 7A-7C are images depicting steps for volumetric filtering and processing for enhanced visualization in accordance with embodiments of the present disclosure;

FIG. 8 depicts images A, B, and C showing MIOCT software interface and manual tracking in accordance with embodiments of the present disclosure;

FIG. 9 shows images of a volumetric time series of a retinal scrape captured with 4D MIOCT;

FIG. 10 illustrates MIOCT recording of the membrane peel along with the corresponding surgical camera frames;

FIG. 11 illustrates 4D MIOCT images of different stages of macular hole surgery;

FIG. 12 depicts images showing dynamic volumetric cyst deformation during membrane peeling visualized with 4D MIOCT;

FIG. 13 shows a detached porcine retina with insertion of a surgical scraper and delivery of subretinal prednisolone acetate in the intervening space between choroid and retina;

FIG. 14 shows representative MIOCT volumetric frames from an imaging period lasting over 1 hour; and

FIG. 15 depicts 4D MIOCT imaging of needle insertion and advancement during deep anterior lamellar keratoplasty (DALK).

### DETAILED DESCRIPTION

For the purposes of promoting an understanding of the principles of the present disclosure, reference will now be made to various embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 36, 37, 38, 39, 40, 41, 42, 43, 44, 45, 46, 47, 48, 49, or 50.

Unless specifically stated or obvious from context, as used herein, the term “about” is understood as within a range of normal tolerance in the art, for example within 2 standard deviations of the mean. The term “about” can be understood as within 10%, 9%, 8%, 7%, 6%, 5%, 4%, 3%, 1%, 0.5%, 0.1%, 0.05%, or 0.01% of the stated value. Unless otherwise clear from context, all numerical values provided herein are modified by the term “about.”

Unless otherwise defined, all technical terms used herein have the same meaning as commonly understood by one of ordinary skill in the art to which this disclosure belongs.

In accordance with embodiments, system and methods are disclosed herein that are configured for to provide four-dimensional (4D) (volumes+time) MIOCT for fast volumetric in vivo imaging of anterior segment and vitreoretinal surgical procedures. In an example, an MIOCT sample arm scanner is integrated with a custom swept-source OCT engine and GPU-based custom software for real time acquisition, processing, and rendering of volumetric images in live anterior segment and retinal human surgeries. Although anterior segment and retinal human surgeries are described in example provided herein, it should be understood that the present subject matter is not so limited and may be otherwise applied to other types of imaging techniques and surgery types. By use of systems and methods disclosed herein, surgical manipulations can be performed in a 3D surgical field. Further, systems and methods disclosed herein can provide volumetric imaging and also display cross-sectional B-scans for improving ophthalmic surgery or other types of surgery.

In accordance with embodiments, the present disclosure provides systems and methods that include or utilize a 4D (volume+time) microscope integrated OCT (MIOCT) for live micron-scale volumetric visualization of microsurgery. In some embodiments, imaging is demonstrated at up to 10 volumes/second.

In accordance with embodiments, the present disclosure provides a 4D MIOCT to elucidate in real time pre-, intra-, and sub-retinal and pre-, intra-, and sub-corneal structural alterations and their interactions with and response to maneuvers with tools and therapeutics and other delivered materials not visible through the microscope.

In accordance with embodiments, the present disclosure provides systems and methods that enable manipulation of each of the different rendering parameters of the real time “view” and the orientation of the viewer from different perspectives and/or within the 3D volume provides unique information which enables performance of techniques and assessment of effects which are not otherwise possible.

In accordance with embodiments, the present disclosure can provide for visualization of 4D MIOCT volume in real time via a video screen or video goggles or other projection to the retina of the viewing operator or surgeon.

Ophthalmic surgery is performed with a microscope that offers only en face visualization. Current intrasurgical imaging with spectral domain OCT is capable of enhancing visualization of surgery but is limited to two-dimensional (2D) real-time imaging.

Also disclosed herein is 4D (volume+time) microscope integrated OCT (MIOCT) system for live micron scale volumetric visualization of microsurgery. The imaging is demonstrated in one example implementation at up to 10 volumes/second, but may be achievable at many times that rate with modifications to the OCT scanning system and “engine”.

In accordance with embodiments, disclosed herein is a stereoscopic heads-up display (HUD) with surgeon control of scanning and display which can be via the surgical microscope oculars, a video screen or video goggles or other projection to the retina of the viewing operator or surgeon.

In surgery, a 4D MIOCT system as disclosed herein can be utilized with a range of standard computer image viewing options (e.g., computer displays) or HUD to elucidate in real time pre-, intra-, and sub-retinal and pre-, intra-, and sub-corneal structural alterations and their interactions with and response to maneuvers with tools and therapeutics and other delivered materials not visible through the microscope. The surface or intra-structural reflectivity of all or selected parts of tools, therapeutics, viscoelastics and other delivered materials may be suitably modified to make them more or less visible to OCT imaging (e.g., small reflective particles added to a fluid to increase OCT signal).

In accordance with embodiments, systems and methods disclosed herein enable manipulation of each of the different rendering parameters of the real time “view” and the orientation of the viewer from different perspectives and/or within the 3D volume. These views can provide unique information to the viewer. Particularly, the viewer may be able to see structures and depths not otherwise available. This may include increasing or decreasing the signal rendered from a specified layer or section of the volume to enable a view of the internal or deeper structures, or combining this with rotation or turning over the volume to optimize the “deeper view” relative to other structures. Anatomic feedback before, during and after maneuvers may be adjusted to expand or distill and optimize information to the surgeon.

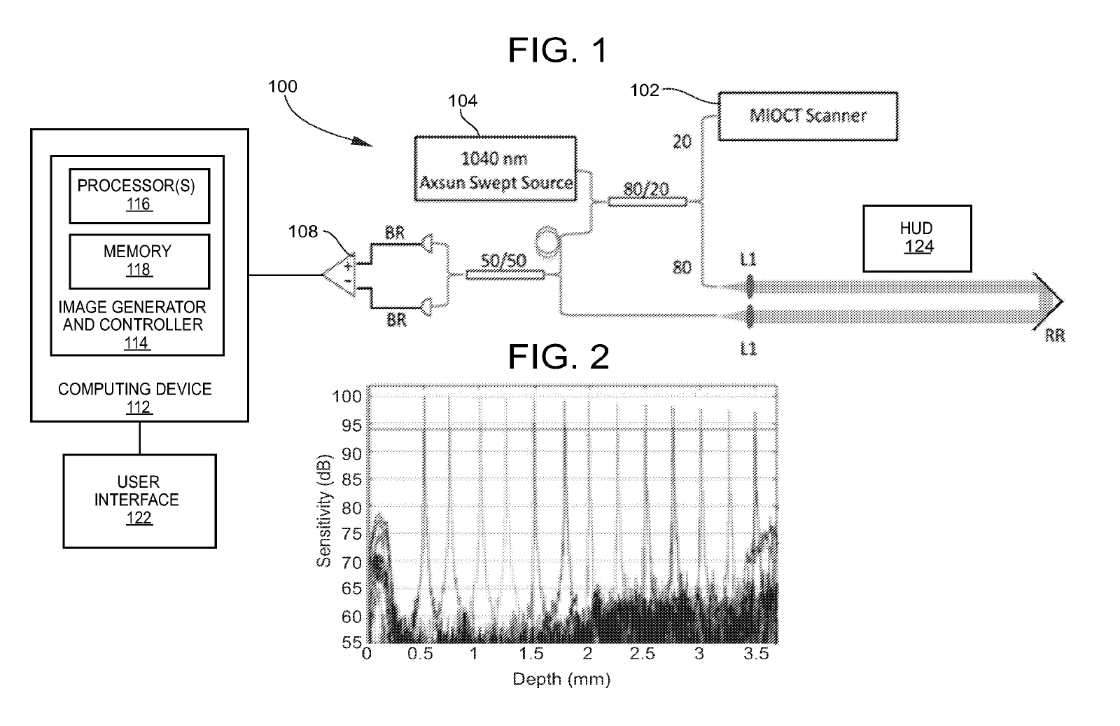

FIG. 1 illustrates a schematic diagram of an example 4D MIOCT system 100 in accordance with embodiments of the present disclosure. The 4D MIOCT system 100 is an image capture system configured to acquire or acquire images of a subject. Particularly, the system 100 includes a sample arm MIOCT scanner 102. A scan head and microscope of the scanner 102 may be co-axial and share the same focal plane. The system 100 also includes a 1040 nm swept-frequency source 104. The swept-frequency source 104 may be a source available from Axsun Technologies of Billerca, Mass. The source 104 may be configured to illuminate a Mach-Zender topology interferometer. The optical signal detection chain includes a balanced photoreceiver and a 1.8 GS/s digitizer, which are represented by component 108. The A-line rate of the SS-OCT system may be 100 kHz, given by the sweep frequency of the source 104. To calibrate acquisition, a laser internal MZI clock may be digitized at 1.8 GS/s to create a re-sampling vector. This resampling vector can, under software control, be interpolated by a factor of two to support extended imaging depth up to zₘₐₓ=7.4 mm for anterior segment imaging, or used without interpolation to achieve an imaging depth of zₘₐₓ=3.7 mm for retinal imaging. In real time during imaging, the photoreceiver output may be digitized at 800 MS/s and re-sampled according to the pre-recorded vector. The axial resolution of the SS-MIOCT system was measured at 7.8 μm and fall off was measured to be 3.9 mm.

FIG. 2 illustrates a graph of a fall off plot showing the sensitivity of the system 100 shown in FIG. 1.

Referring again to FIG. 1, the system 100 includes capturing cross-section images of a subject, such as structures of an eye 110. The captured images may be received by a computing device 112 that is operably connected to the photoreceiver and digitizer 108. The computing device 112 may be a desktop computer, a laptop computer, a tablet computer, a smartphone, or the like configured to implement the functionality described herein. Particularly, the computing device 112 may include an image generator and controller 114 configured to implement functionality described herein in accordance with embodiments of the present disclosure. The image generator and controller 114 may be implemented by hardware, software, firmware, or combinations thereof. For example, the image generator and controller 114 may include one or more processors 116 and memory 118. The memory 118 may store instructions for execution by the processor(s) 116 for implementing the functionality disclosed herein. Particularly, the image generator and controller 114 can generate a stereoscopic left image and right image of the subject based on the received cross-sectional images. Further, the image generator and controller 114 can control the display of the stereoscopic left image and the right image in a display of a microscope system 120. Additional details of the implementation of these functions are disclosed herein.

In accordance with embodiments, a user interface 122 may be operably connected to the computing device 112 for receipt of user input and for the presentation of data, information, and images to an operator, such as a surgeon and/or other healthcare practitioner. In an example, the image generator and controller 114 implemented 4D MIOCT control software, which can provide for operator choice of the display of a variety of lateral OCT scan patterns, including raster-scanned volumes with arbitrary numbers of A-scans per B-scan and B-scans per volume. Volumetric acquisition rates evaluated in human and simulated surgeries ranged from 1.8 volumes/sec (for 2624×544×100 voxels) for high quality visualization and archiving, up to 10 volumes/sec (for 2624×100×100 voxels) for real time instrument tracking. The system 100 was employed on consented patients undergoing macular and anterior segment surgeries.

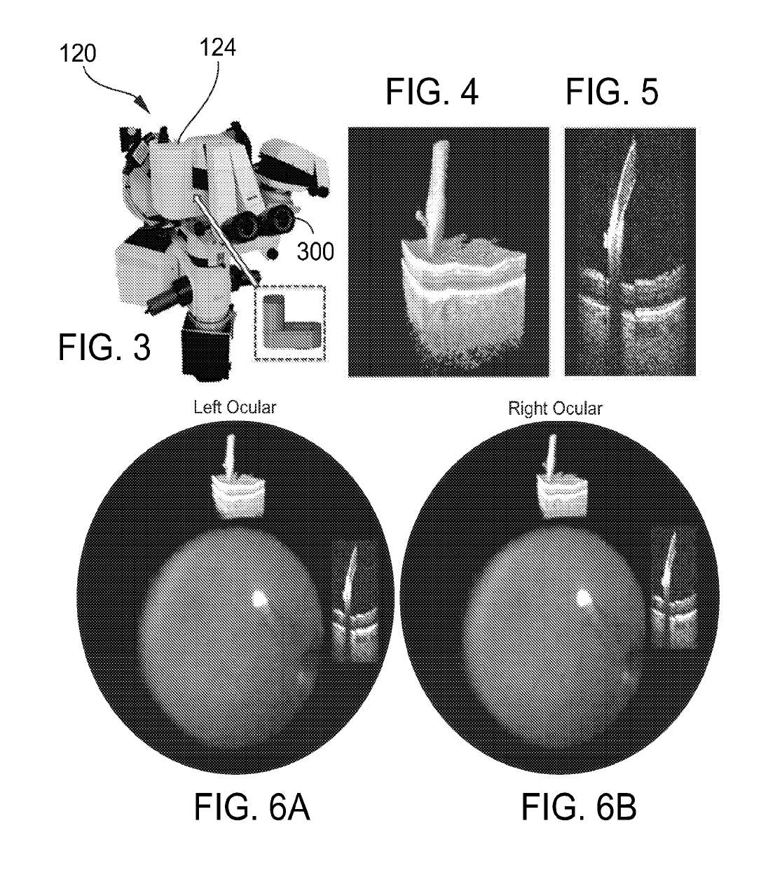

In accordance with embodiments, a HUD 124 may be integrated with the microscope system 120. FIG. 3 is an image of an example microscope system 120 including an MIOCT scanner 102 and the HUD 124. In this example, the HUD 124 is a dual-channel HUD that allows simultaneous projection of MIOCT volumes rendered from different perspectives and projected in real-time into surgical oculars 300. The rendered perspectives enable stereoscopic visualization of the volumes. The location of data projected within the oculars 200 was controlled by the 4D MIOCT operator to ensure that the surgical field was not obstructed. The operator may also project arbitrarily chosen B-scans, MIPs, and other relevant surgical data using the HUD. The user interface 122 may include a foot-operated joystick or foot pedal configured to receive user input for changing a perspective of the 3D image from one perspective to another perspective. For example, a surgeon may use the foot pedal to change the orientation of the MIOCT volume during image acquisition. In embodiment, the generated 4D MIOCT data may also be displayed in real-time on a wall-mounted, high-definition display or the like in the operating suite to facilitate data analysis by other surgical staff. The inset in the lower right portion of FIG. 3 shows a model of the HUD unit enclosure.

FIG. 4 is an image showing an MIOCT volume generated in accordance with embodiments of the present disclosure. FIG. 5 is an image of a B-scan acquired in accordance with embodiments of the present disclosure. A HUD in accordance with embodiments of the present disclosure may project the images shown in FIGS. 4 and 5 into operating microscope oculars to enable concurrent visualization of MIOCT data and the operating microscope view. FIGS. 6A and 6B are images of a left ocular view and a second ocular view respectively after projection of MIOCT data. In this example, the MIOCT B-scans and volumes are placed in the periphery of the operating microscope field of view to avoid obstruction of the surgical field. Volumes rendered at different perspectives were projected into the right and left ocular to enable stereoscopic visualization of 4D MIOCT data. The 4D data provide feedback on orientation of tool to adjacent structures or tissues and structures from within the vitreous cavity deep into the sclera.

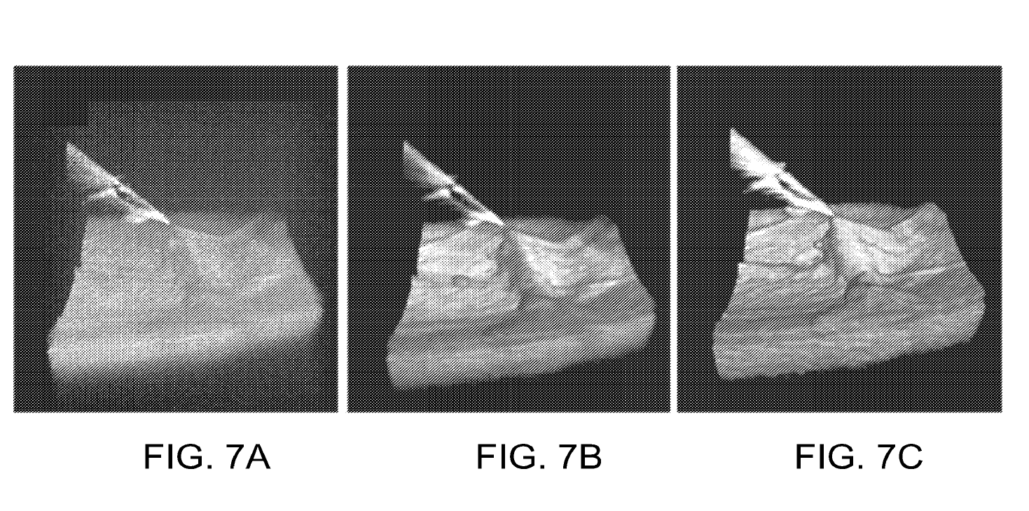

In accordance with embodiments, software enabled real-time acquisition, processing, and rendering of volumetric data sets acquired at 100 kHz line rates. The software was written in C/C++ and comprised three concurrent threads; a data collection thread, a data processing and rendering thread, and a display thread. The data collection thread communicated with the acquisition card and collected 4000 spectral samples of data for each A-scan. 16 B-scans were processed at a time through the use of custom GPU code written in CUDA and executed on a GTX Titan (NVIDIA; Santa Clara, Calif.). Once the data was processed, three different views of the data were created: a volumetric view, a single B-scan view, and a maximum intensity projection (MW) en face view. The volumetric view may be created by filtering the processed data with a 3×3×3 median filter, followed by filtering each B-scan with a 5×5 two-dimensional Gaussian filter. The resulting volume may be rendered to a two dimensional image using ray casting, edge enhancement, and depth-based shading as shown in FIGS. 7A-7C. The display thread may use OpenGL to display the acquired live volume, a single B-scan pre-selected from the volume by the user, and the MW of the volume data. The GPU-based software also incorporated “stream saving” to save each volumetric dataset immediately after acquisition without user input, enabling continuous 4D recording.

FIGS. 7A-7C are images depicting steps for volumetric filtering and processing for enhanced visualization. Particularly, FIG. 7A shows an unfiltered volume of a surgical field. FIG. 7B shows the volume after median and Gaussian filtering. FIG. 7C shows the volume after edge enhancement and depth-based lighting. The MIOCT volume shown was captured during porcine eye surgery. Retinal vasculature, which was minimally visible in FIG. 7A (linear ridges from left to right), are prominently shown in FIG. 7C as were the cross-sectional layers at the leading border.

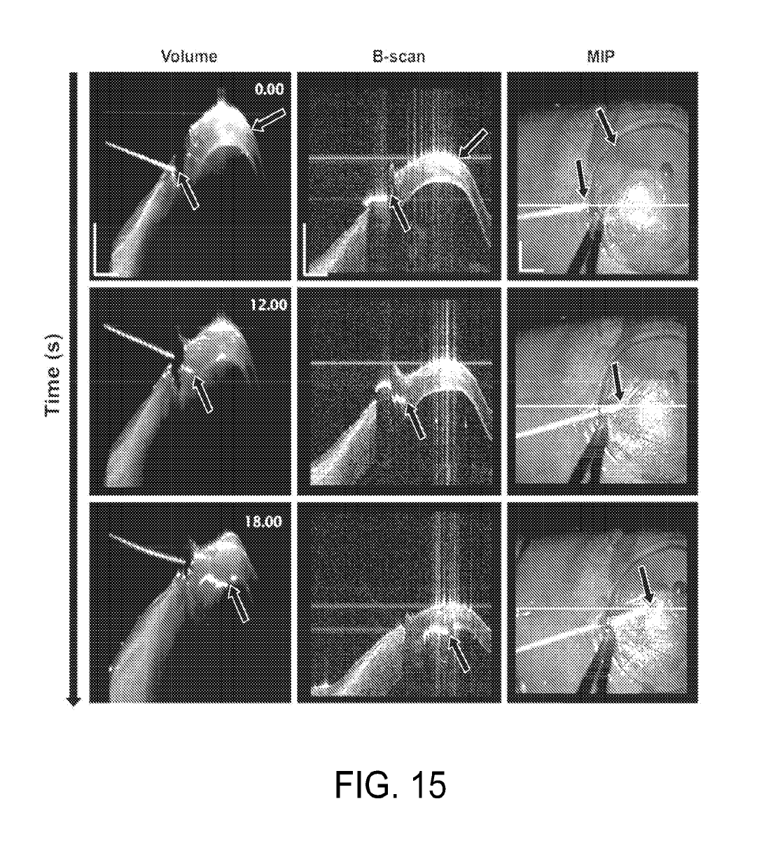

In accordance with embodiments, an MIOCT scan may be rotated arbitrarily during surgery to align the B-scan axis to a particular maneuver, tool, or region of interest. For example, this feature was often used to optimize view of traction to retina and to visualize needle advancement in DALK shown in FIGS. 9 and 15. By digitizing the optical clock provided by the source and resampling, a variable axial scan length between 3.7-7.4 mm can be achieved. Furthermore, mixed mode volumes, in which only the B-scan of interest arbitrarily chosen within the OCT field of view was densely sampled and averaged while the rest of the data was sparsely sampled to preserve a fast volumetric rate. The typical posterior segment protocol consisted of 3.7 mm axial imaging range and 300 A-lines/B-scan by 100 B-scans per volume, resulting in a volumetric rate of 3.33 Hz with maximum latency of 0.3 seconds. The anterior segment imaging protocol consisted of 7.4 mm axial imaging range and 500 A-lines/B-scan and 100 B-scan per volume, resulting in a volumetric rate of 2 Hz with a maximum latency of 0.5 seconds. The volumetric acquisition rate for 4D MIOCT imaging is ultimately limited by the laser sweep frequency (100,000 A-scans/second), and trades off with the lateral sampling density (number of A-scans per volume) desired for particular applications. Faster frame rates can be achieved by further down-sampling. It was determined that sampling at 120 A-scans/B-scan and 120 B-scans/volume can still yield high quality volumetric renders at ˜7 volumes per second while still preserving sample structural information in single cross-sectional images. Furthermore, isotopic sampling yielded orthogonally oriented B-scans of similar quality. Series of radially oriented B-scans centered on structures of interest (e.g., macular holes) were also acquired (not shown). To demonstrate 4D MIOCT imaging at ˜10 volumes per second for example, the number of B-scans can be reduced to 80 while preserving 120 A-scans/B-scan.

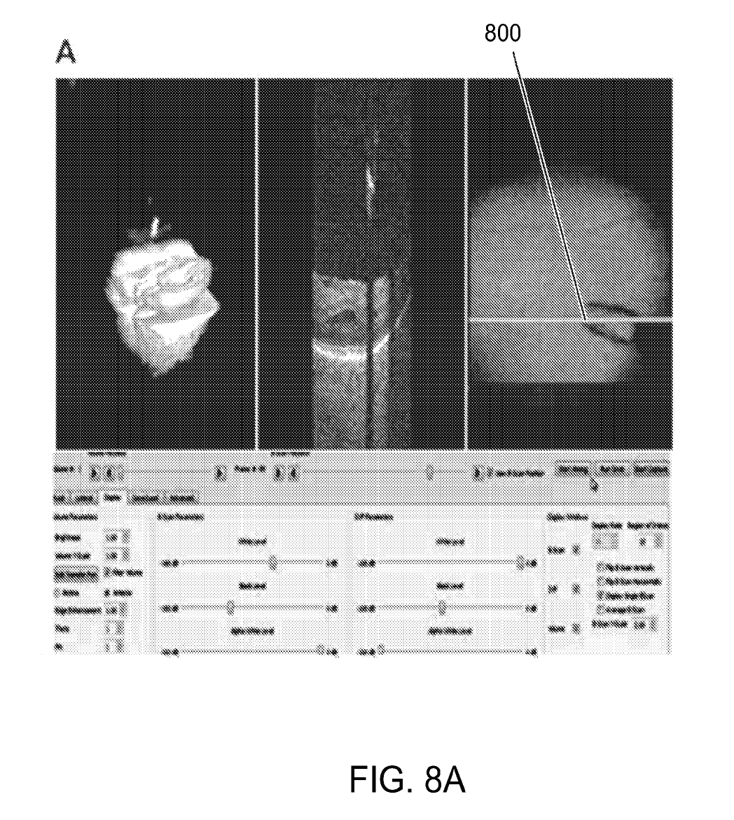

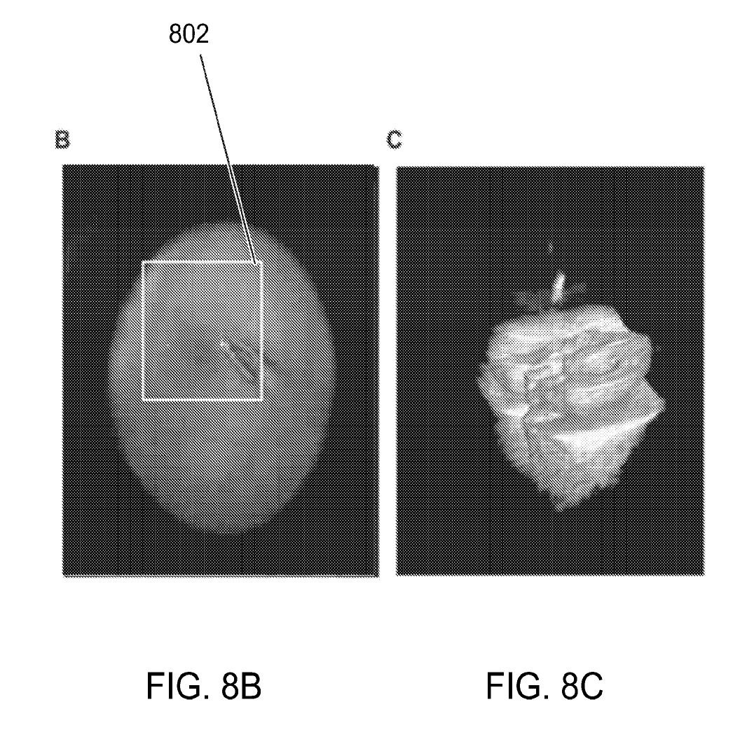

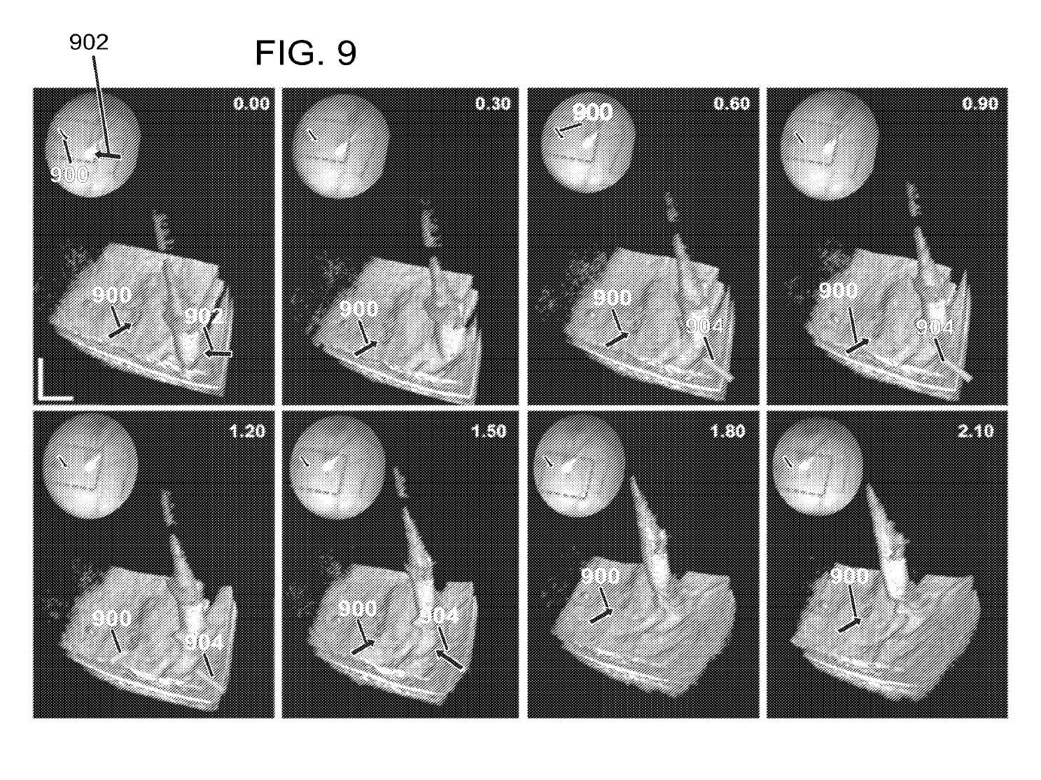

In an experimental setup, an MIOCT software interface included 3 monitors and was controlled by a dedicated operator during surgery. For example, FIG. 8 depicts images A, B, and C showing MIOCT software interface and manual tracking in accordance with embodiments of the present disclosure. The first monitor shown in image A of FIG. 4 displays controls for the OCT scan parameters, saving and loading data, and adjustable MIP (with a line 800 denoting the location of the displayed B-scan), volume, and B-scans viewing windows. The second monitor shown in image B of FIG. 8 displays a feed from the surgical camera in which a rectangle 802 delineates the MIOCT lateral field of view. Clicking and dragging this rectangle 802 resulted in lateral translation of the MIOCT scan to an arbitrary location on the surgical field. This manual-tracking feature was especially useful when imaging features in motion to ensure that the region of interested was always centered on the OCT field of view. Reorienting the plane of scan so that it was parallel or perpendicular to the axis of an instrument, or aligned at a specific angle relative to motion of tissue or tools, improved visualization of structures of interest. The third monitor shown in image C of FIG. 4 mirrors what was displayed in the HUD and enabled the OCT operator to control data content and location of the projected data in the surgeon's field of the view.

4D MIOCT imaging was performed in 47 human surgeries, including vitreoretinal and anterior segment surgeries. During imaging, MIOCT optical power on the eye was below 1.7 mW and the intraocular visible illumination was reduced by 20% to maintain the total irradiance to below the maximum permissible exposure for ocular illumination. Representative data from four vitreoretinal cases and one anterior segment case are shown and discussed herein. All representative data shown was rendered (including filtering, lighting and edge enhancement) and displayed in real-time during surgery. All videos provided in supplementary materials play back at the real-time 4D MIOCT volumetric acquisition rate. A microscope-integrated dual-channel HUD enabled stereoscopic visualization of 4D MIOCT via the surgical oculars.

Vitreoretinal microsurgery involves restoration of micro-architectural retinal alterations that arise from pathologic conditions. In one such condition, an epiretinal membrane (ERM) can proliferate and contract on the surface of the retina, causing visual distortion and loss of central vision. Full thickness macular holes can also result from traction from the vitreous gel, from contraction of these pathologic ERMs, or from intrinsic traction from the native internal limiting membrane (ILM). Microsurgical forceps and/or scrapers can be used to peel these pathologic and/or native membranes to relieve underlying retinal contraction and close the retinal defect.

4D MIOCT can be used for enhanced real-time visualization during surgical repair of a full-thickness macular hole. FIG. 9 shows images of a volumetric time series of a retinal scrape captured with 4D MIOCT. The corresponding surgical camera frames are located in the upper-left of each OCT image. Time stamps (in seconds) are located in the upper right and referenced to the first frame. The black dashed box in the surgical camera frames denotes the MIOCT field of view. Arrows 900 denote the location of macular hole in both the operating microscope and MIOCT images. Arrows 902 denote the location of the tip of the scraper in the first frame of both the operating microscope and MIOCT images. Arrows 904 point to a retinal depression caused by the maneuver that was only visible in the 4D MIOCT data. The scale bars are 1 mm. The volumetric data was acquired, processed, and displayed at 3.3 volumes/second. FIG. 9 shows excerpts from live MIOCT visualization of a diamond dust-coated surgical scraper brushing against the retinal surface around a full-thickness macular hole. The corresponding frames from a surgical camera that records the surgeon's view through the operating microscope are shown next to each MIOCT volume. The scraper 902 was visualized in both the operating microscope view as well as in the MIOCT view. The macular hole was also clearly visualized in the MIOCT view, while it was more difficult to identify using the operating microscope alone (shown by arrows 900). Furthermore, 4D MIOCT enabled visualization of 3D features in the surgical field that were not evident in the operating microscope view, such as an apparent retinal depression caused by the scraper (arrows 904).

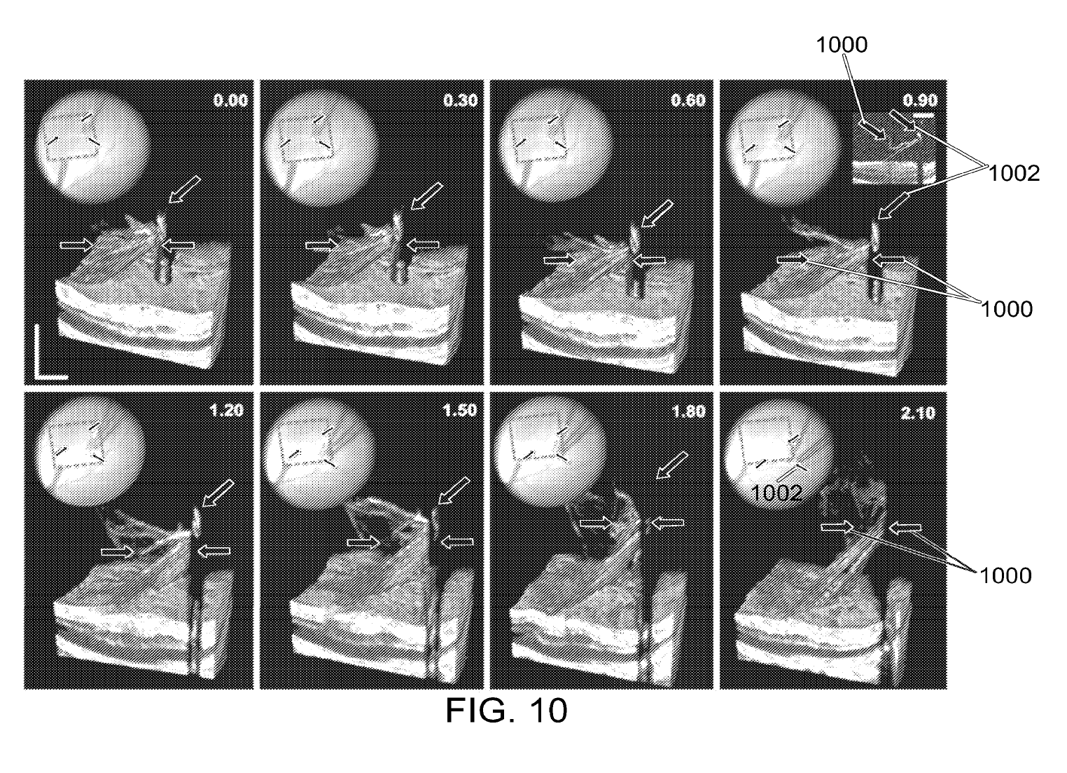

4D MIOCT also improved real-time visualization of surgical peeling of ERMs, which are typically tens of microns thick and challenging to visualize through the operating microscope alone. FIG. 10 illustrates MIOCT recording of the membrane peel along with the corresponding surgical camera frames. In the operating microscope view, these thin membrane sheets and the membrane/retina interface are difficult to visualize due to the lack of contrast between the membranes and background tissue. 4D MIOCT enabled clear visualization of the ERM (arrows 1000) as it was peeled using surgical forceps. Although the entire forceps were not visible in OCT, the tips and the tissue-tool interface (arrows 1002) were clearly visualized in three dimensions along with the interface between the healthy retinal tissue and the diseased membrane. Moreover, the exact depth position of the forceps tip relative to the retinal surface was directly visible in MIOCT while it could only be inferred indirectly using the stereo view and instruments shadows visible in the operating microscope.

More particularly, FIG. 10 shows volumetric time series of an epiretinal membrane (ERM) peel in vitreoretinal surgery using 4D MIOCT. The corresponding surgical camera frame is located in the upper-left of each OCT image. Time stamps (in seconds) are provided in the upper-right and referenced to the first frame. The black dashed box in the surgical camera frames denotes the MIOCT field of view. Arrows 1000 denote the location of the ERM in the surgical camera and MIOCT frames. Arrow 1002 denotes the location of the tip of the surgical forceps in the surgical camera and MIOCT frames. Note that only the tip of the surgical forceps is visible in the MIOCT view due to lack of OCT light backscattered from the rest of the metallic instrument. The membrane peel is readily visualized in the MIOCT view while it is translucent in the surgical camera view. MIOCT also allows for precise depth localization of the tip of the surgical forceps relative to the retinal surface. The inset in the upper-right of frame 0.90 shows a single-frame B-scan located at the tool/ERM interface. The scale bars are 1 mm. The volumetric data was acquired, processed, and displayed at 3.3 volumes/second.

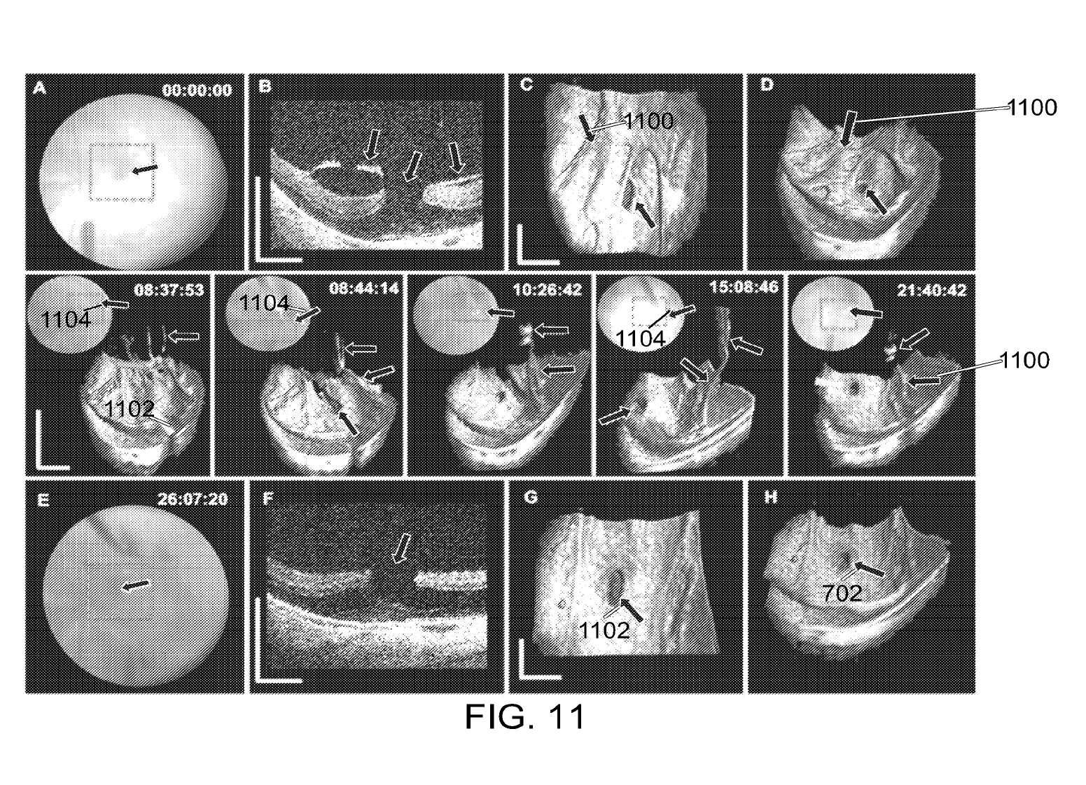

4D MIOCT was also be used to obtain high-resolution volumes and line scans at pauses in surgery to confirm anticipated surgical outcomes and evaluate for complications. For example, FIG. 11 illustrates 4D MIOCT images of different stages of macular hole surgery. The black dashed box in the surgical camera frames denotes the MIOCT field of view. Time stamps are in minutes:seconds:milliseconds and referenced to the first frame. Images A-D show the surgical camera view (A), B-scan (B), and volumes rendered at difference perspectives (C-D) at time 00:00:00. A partial thickness macular hole can be caused by contraction of pathologic ERM and/or ILM, and the primary surgical goal is to remove ERM and ILM to relieve retinal surface tension causing cystoid structures and decrease in visual acuity. Pre-maneuver MIOCT images (images B and C of FIG. 11) demonstrated enhanced visualization of the ERM/ILM (arrows 1100) around the partial thickness macular hole (arrows 1102) compared to the surgical microscope view (image A of FIG. 11). The B-scan provided exquisite detail of ERM relative to the retinal surface and important feedback that there was reflective retinal tissue within the hole (below arrow 1102) verifying that it did not extend full thickness through the retina (image B of FIG. 11). Volumes rendered at different perspectives (controlled by the surgeon in real-time) revealed (images C and D of FIG. 11) the complex 3D micro-architecture of the ERM. Complete surgical peeling and aspiration of the ERM and ILM was recorded with 4D MIOCT. See FIG. 11, 08:37:53-21:40:42. The corresponding surgical camera frames were also captured. The three-dimensional tissue/tool interaction was clearly visible in the volumes but difficult to discern with the surgical microscope alone, even though a common technique of staining the surface ILM tissue with indocyanine green dye was used to improve the surgeon's visualization through the microscope. The surgeon viewed the post maneuver MIOCT volumetric images and B-scans were used to verify that the ERM was successfully peeled (images E-H of FIG. 11) and that the deep retinal tissue remained intact and thus the lesion had not progressed to a full-thickness hole during surgery (under arrow in image F of FIG. 11). Furthermore, the micro-architectural alterations between the pre and post maneuver time points were difficult to visualize through the surgical microscope but were readily apparent especially in the MIOCT volumes (images G and H of FIG. 11).

The pre maneuver MIOCT images shown in FIG. 11 reveal the complex 3D micro-architecture of the ERM not appreciable through the operating microscope. Representative MIOCT volumes from various surgical maneuvers from time 08:37:53-21:40:42 are shown. Arrow 1104 denotes the tip of the surgical scraper and the purples arrows denotes the tip of the vitrector (used for cutting and aspirating ERM). The volumes show the surgeon alternating between peeling (08:37:53-08:44:14, 15:08:46) and cutting/aspirating ERM (10:26:42, 21:40:42). The tissue/tool interaction is clearly visualized in the MIOCT volumes. Images E-H show the surgical camera view (E), B-scan (F), and volumes rendered at difference perspectives (G-H) acquired after completion of maneuvers (26:07:20) with retinal blood vessels visible as linear elevations at the surface. The post maneuver 4D MIOCT images reveal prominent micro-architectural alterations not readily apparent through the microscope and were used to verify successful peeling of ERM. Scale bars are 1 mm. Volumetric images were acquired at 3.33 volumes/second.

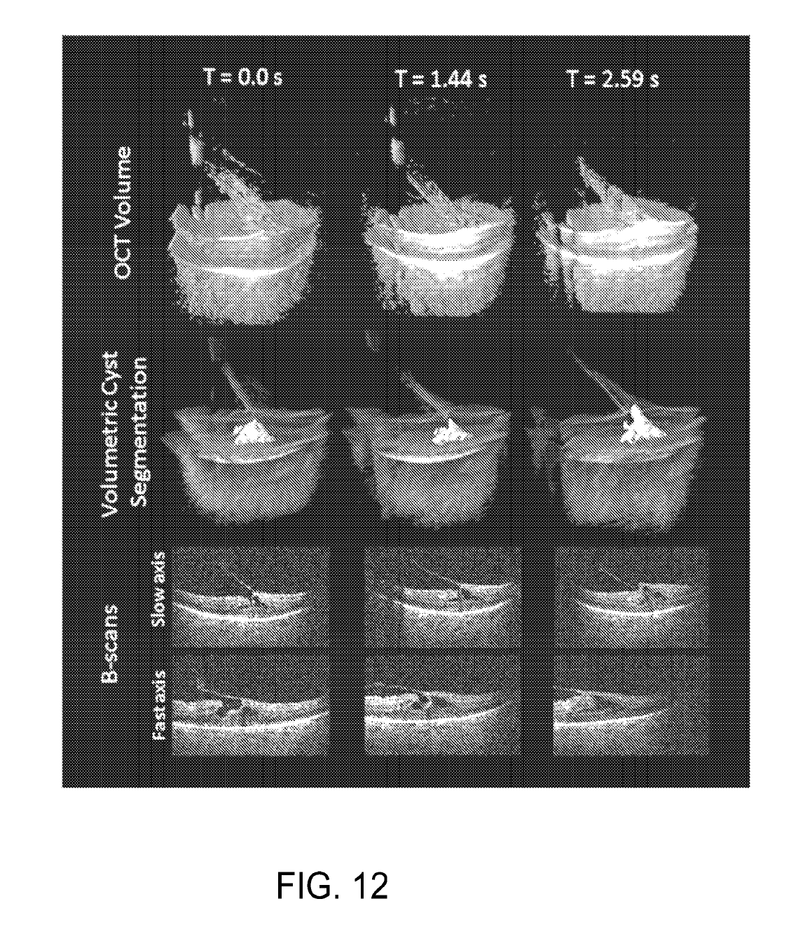

4D MIOCT was also be used to evaluate volumetric deformation of retinal cysts during membrane peeling. Volumetric images were acquired at 6.94 vols/second (120 A-lines/B-scans, 120 B-scans/volume) during lamellar hole repair. Retinal cysts, not visible through the surgical microscope, were manually segmented in post-processing in the volumes; however, this is an example of segmentation that can be completed and displayed in near real time to guide surgical decision-making. The segmented cysts were artificially designated high intensity values in the B-scans to facilitate visualization by manipulating the voxel intensity histogram of the volumes. FIG. 12 depicts images showing dynamic volumetric cyst deformation during membrane peeling visualized with 4D MIOCT. Referring to FIG. 12, retinal tissue was made translucent while artificially coloring (coloring not shown) the segmented cysts in the middle row to enhance visualization. FIG. 12 also shows the volumes before histogram manipulation. In addition, orthogonally oriented B-scans show the cysts in cross-section. The volumetric images, after histogram manipulation, show the cysts deformation due to traction from the membrane peel.

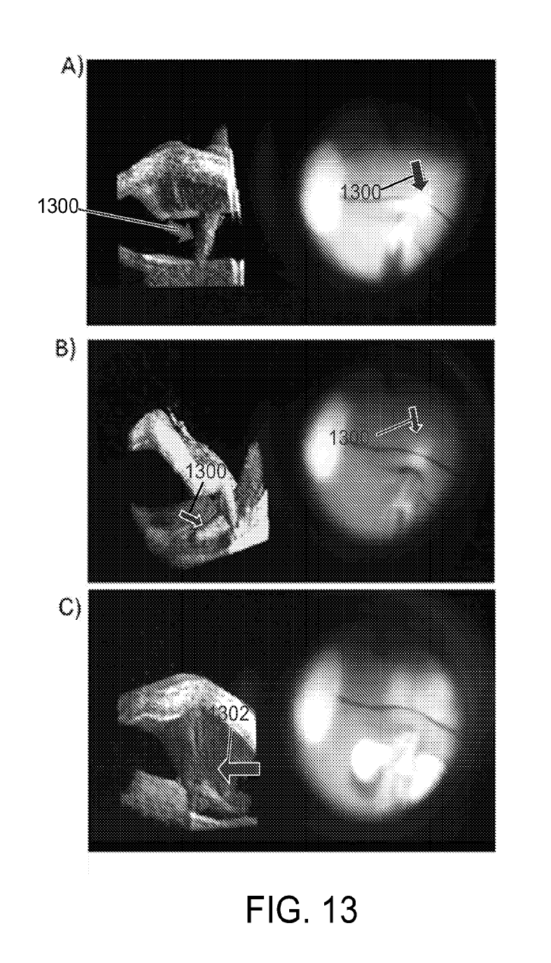

Moreover, 4D MIOCT was used to visualize separation of the retina and structures, materials and tools between retina and choroid in cases treating retinal detachments or in experiments where separation of the retina from the underlying retinal pigment epithelium was purposefully created for the trial delivery of OCT-reflective liquid which could model injection of stem cells of a type reflective on OCT or modified to make them visible on OCT. The 3D location of the subretinal instrument and the injected material on OCT far exceeds the poor view into the subretinal space with the traditional surgical microscope vie. FIG. 13 shows a detached porcine retina with insertion of a surgical scraper and delivery of subretinal prednisolone acetate in the intervening space between choroid and retina. More particularly, FIG. 13 depicts images of 4D MIOCT for visualizing the intervening space between retina and choroid during porcine retinal detachment. Images A and B show volumes from different time points. The surgeon manipulated the volumetric orientation in real-time to enhance tool visualization underneath retina. Arrows 1300 denote the tip of the surgical instrument. Image C shows sub-retinal triamcinolone acetate injection (arrow 1302). As evident, the axial location of the surgical tip or triamcinolone acetate within the subretinal space can only be localized accurately in the MIOCT volumes. The 4D MIOCT volumes as well as the corresponding surgical camera frames are shown. As evident, the en face view provided by the surgical camera cannot be used to determine the position of the instrument tip inside tissue. Because the retina transmits light at the wavelengths of the OCT, the volumetric images can be used by the surgeon to readily determine the location of the surgical tip within tissue. Furthermore, the surgeon can control the perspective and viewpoint of the rendered volumetric images in real time to provide visualization within or beneath the retina or other human or animal tissues. This provides a unique method for controlled monitoring in multiple dimensions and from different perspectives of 1) the delivery of instrumentation, laser energy, therapeutics and cells and 2) the manipulation of materials, tissue, cells, instrumentation.

Anterior eye surgeries are among the most commonly performed surgeries worldwide. The focus of this section is on corneal transplantation, in which at least a portion of the patient's diseased cornea is replaced with a donor corneal graft. In a full-thickness corneal transplant, or penetrating keratoplasty, the patient's entire cornea is replaced and a graft must be sutured in its place.

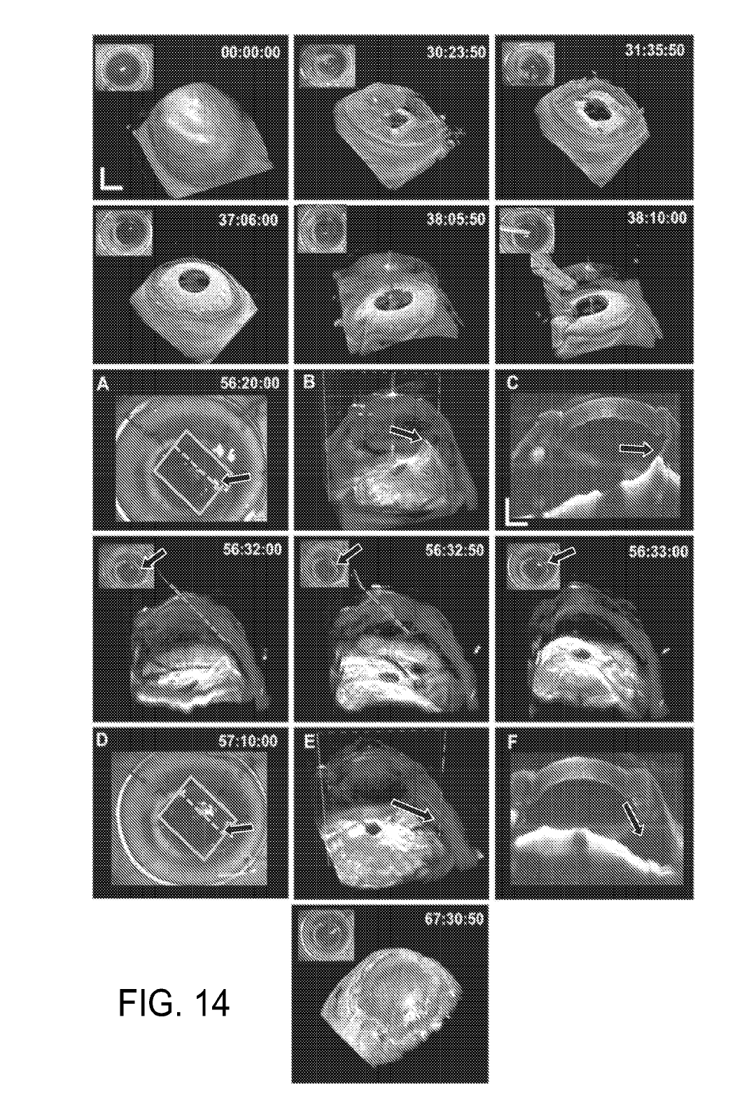

4D MIOCT imaging was performed in a penetrating keratoplasty procedure to visualize replacement of the host cornea with the donor graft. Using live volumetric recording, the entire corneal transplant was recorded with 4D MIOCT in ˜5 minute segments. FIG. 14 shows representative MIOCT volumetric frames from an imaging period lasting over 1 hour. The different stages of the corneal transplantation were clearly visualized. First, the native cornea was dissected and removed (FIG. 14, 30:23:50-37:06:00). Removal of the host cornea was readily visible in 4D MIOCT. Next, the corneal graft was inserted and sutured into the native tissue (FIG. 14, 38:05:50-38:10:00). The graft was also visible in MIOCT. Because of the difference of back-scattered light intensities, the iris appeared much brighter than the corneal tissue/graft in the MIOCT images. The difference in intensities allowed intensity-based thresholding to enhance MIOCT visualization of structures beneath the corneal graft (FIG. 14, images A-C). At this time (FIG. 14, 56:20:00), incarceration of the iris became visible only in the MIOCT images. The surgeon was unable to localize the incarcerated iris using only the en face surgical microscope view (FIG. 14, image A). If the incarcerated iris were not resolved, this could have led to post-operative complications such as wound leakage, local corneal endothelial cell loss, increased inflammation, and glaucoma. Using MIOCT for localization guidance, the surgeon was able to direct a cannula (dashed line) and inject viscoelastic between the iris and corneal graft to release the iris (FIG. 14, 56:32:00-56:33:00). Further evaluation using MIOCT revealed resolution of the incarcerated iris with clear intervening space between the iris and cornea (FIG. 10, images D-F) and subsequently, the donor graft was secured to the host (FIG. 14, 67:30:50).

Referring to FIG. 14, the figure depicts 4D MIOCT imaging of corneal transplantation surgery. Volumetric images were recorded over a period of ˜1 hour, covering all steps of the transplantation procedure. Time stamps are in minutes:seconds:milliseconds. Volumetric images were acquired at 2 volumes/second. Scale bars are 1 mm. The corresponding surgical camera frames are shown as well. Representative volumetric frames of each step in the procedure are shown (00:00:00-67:30:50). At time 00:00:00, the intact cornea is illustrated. From time 30:23:50 to 37:06:00 the native cornea was dissected and excised. From time 38:05:50 to 38:10:10, the corneal graft was sutured into place. Before finishing the graft suturing, at time 56:20:00 MIOCT volumetric images revealed iris abnormally incarcerated in the donor-host interface (arrows in the first row of images A-C). Images A-C of FIG. 14 show the surgical camera frame, MIOCT volumetric image, and B-scans, respectively. The location of the MIOCT volume and B-scan are denoted on the surgical camera view by the light square and dashed line, respectively. The location of the B-scan denoted by the white rectangle in the volume view, was chosen to provide the surgeon with cross-sectional visualization of the abnormal iris. From time 56:32:00 to 56:33:00 (Movie S3), the surgeon was able to direct a cannula (dashed line on the MIOCT volumes and arrows in the second row of images A-C on the surgical camera frames) to the site of the lesion using MIOCT guidance and inject viscoelastic to resolve the incarcerated iris. Images D-F show the surgical camera frame, MIOCT volume, and B-scan, respectively after injection of viscoelastic. The MIOCT volume and B-scan revealed that the iris was successfully released (arrows in images D-F) while the surgical microscope was not able to provide any information. The graft suturing was completed at time 67:30:50.

Use of OCT during anterior segment surgery has been limited and others have noted the need for further development before practical real-time use. In an example implementation, the utility of 4D MIOCT was demonstrated for monitoring a corneal transplant and providing guidance of select maneuvers. This MIOCT technology has also been used in deep anterior lamellar keratoplasty (DALK) and Descemet's stripping endothelial keratoplasty (DSEK) procedures (FIG. 15), in which either the anterior or posterior cornea is excised while leaving healthy native cornea intact. These procedures require precise axial localization of tools within the corneal stroma and the graft/host cornea interface, both of which are difficult to obtain with the operating microscope but are readily achievable with real-time volumetric MIOCT recording. 4D MIOCT feedback using the HUD could increase surgical efficiency and accuracy in these procedures.

FIG. 15 depicts 4D MIOCT imaging of needle insertion and advancement during deep anterior lamellar keratoplasty (DALK). Volumes, B-scans, and maximum intensity projection (MIP) (en face OCT images) are shown at 3 different time points. Times stamps are in seconds. The horizontal line in the MIP denotes the location of the B-scan. The goal of the maneuver is to separate the anterior 90% of cornea from Descemets's membrane (posterior 10%) by injecting an air bubble at the interface. Needle insertion requires micron-scale axial precision to prevent penetration into the anterior segment. Unlike the surgical microscope, MIOCT generates micron-scale volumetric images to provide direct visual feedback of the needle location within cornea. Volumetric images were acquired at 2 volumes/second with 500 A-lines/B-scan. Scale bars are 1 mm.

Disclosed herein is real-time, volumetric, micron-scale visualization of human ophthalmic microsurgery. A prototype 4D MIOCT system was used in 47 human surgeries to image a variety of vitreoretinal and corneal surgical maneuvers and elucidated structural information in the surgical field that was not evident in the operating microscope view. Towards MIOCT-guided microsurgery, a custom stereoscopic HUD was developed to enable concurrent visualization of the MIOCT and operating microscope views by the surgeon. 4D MIOCT provided real-time, tomographic structural information that may be used to evaluate maneuvers and help guide microsurgery.

In accordance with embodiments of the present disclosure, orientation and/or positioning of the display of images, such as a 3D images, as disclosed herein may be controlled by an operator by any suitable technique. For example, any suitable user interface may be used to input commands for controlling a view of a 3D image. One example is the use of a foot pedal for inputting commands. This technique can be advantageous because the operator's hands may be free for operating other equipment.

The various techniques described herein may be implemented with hardware or software or, where appropriate, with a combination of both. Thus, the methods and apparatus of the disclosed embodiments, or certain aspects or portions thereof, may take the form of program code (i.e., instructions) embodied in tangible media, such as floppy diskettes, CD-ROMs, hard drives, or any other machine-readable storage medium, wherein, when the program code is loaded into and executed by a machine, such as a computer, the machine becomes an apparatus for practicing the presently disclosed subject matter. In the case of program code execution on programmable computers, the computer will generally include a processor, a storage medium readable by the processor (including volatile and non-volatile memory and/or storage elements), at least one input device and at least one output device. One or more programs may be implemented in a high level procedural or object oriented programming language to communicate with a computer system. However, the program(s) can be implemented in assembly or machine language, if desired. In any case, the language may be a compiled or interpreted language, and combined with hardware implementations.

The described methods and apparatus may also be embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, a video recorder or the like, the machine becomes an apparatus for practicing the presently disclosed subject matter. When implemented on a general-purpose processor, the program code combines with the processor to provide a unique apparatus that operates to perform the processing of the presently disclosed subject matter.

Features from one embodiment or aspect may be combined with features from any other embodiment or aspect in any appropriate combination. For example, any individual or collective features of method aspects or embodiments may be applied to apparatus, system, product, or component aspects of embodiments and vice versa.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims. One skilled in the art will readily appreciate that the present subject matter is well adapted to carry out the objects and obtain the ends and advantages mentioned, as well as those inherent therein. The present examples along with the methods described herein are presently representative of various embodiments, are exemplary, and are not intended as limitations on the scope of the present subject matter. Changes therein and other uses will occur to those skilled in the art which are encompassed within the spirit of the present subject matter as defined by the scope of the claims.

What is claimed:

1. A method comprising:
capturing a plurality of cross-sectional images of a subject;
generating a stereoscopic left image and right image of the subject based on the cross-sectional images; and
displaying the stereoscopic left image and the right image in a display of a microscope system.

2. The method of claim 1, wherein the subject comprises an eye.

3. The method of claim 1, wherein the subject is a retina of an eye.

4. The method of claim 1, wherein capturing a plurality of cross-sectional images of a subject comprises capturing a plurality of B-scan images of the subject.

5. The method of claim 1, wherein capturing a plurality of cross-sectional images comprises using an optical coherence tomography (OCT) technique for capturing the cross-sectional images.

6. The method of claim 1, wherein generating a stereoscopic left image and right image comprises:
filtering the left and right images; and
applying an edge enhancement and depth-based light technique to the filtered images.

7. The method of claim 1, wherein the display of microscope system comprises a left ocular and a right ocular, and
wherein displaying the stereoscopic left image and the right image comprises displaying the stereoscopic right image and the right image in the left ocular and the right ocular, respectively.

8. The method of claim 1, wherein displaying the stereoscopic left image and the right image comprises displaying the stereoscopic left image and the right image in one of a heads-up display, a video screen, and video goggles.

9. The method of claim 1, wherein displaying the stereoscopic left image and the right image comprises displaying the stereoscopic left image and the right image of the subject from a first perspective, and
wherein the method further comprises:
receiving input via a user interface for changing the display of the subject to a second perspective different than the first perspective; and
in response to receipt of the input:
generating another stereoscopic left image and right image of the subject based on the cross-sectional images; and
displaying the other stereoscopic left image and the right image in the display of the microscope system.

10. The method of claim 1, further comprising displaying at least one of the cross-sectional images in the display of the microscope system.

11. The method of claim 10, wherein the user interface comprises a foot pedal controller.

12. The method of claim 1, wherein the plurality of cross-sectional images are a first plurality of cross-section images,
wherein the stereoscopic left image and the right image are a stereoscopic first left image and a first right image;
wherein capturing a plurality of cross-sectional images comprises capturing the first plurality of cross-sectional images within a first time period, and
wherein the method further comprises:
capturing a second plurality of cross-sectional images of the subject; and
generating a stereoscopic second left image and second right image of the subject; and
displaying the stereoscopic second left image and second right image in the display at a time different than the display of the stereoscopic first left image and the first right image.

13. A system comprising:
an image capture system configured to capture a plurality of cross-sectional images of a subject;
an image generator and controller configured to:
generate a stereoscopic left image and right image of the subject based on the cross-sectional images; and
display the stereoscopic left image and the right image in a display of a microscope system.

14. The system of claim 13, wherein the subject comprises an eye.

15. The system of claim 13, wherein the subject is a retina of an eye.

16. The system of claim 13, wherein the image capture system is configured to capture a plurality of B-scan images of the subject.

17. The system of claim 13, wherein the image capture system is configured to use an optical coherence tomography (OCT) technique for capturing the cross-sectional images.

18. The system of claim 13, wherein the image generator and controller are configured to:
filter the left and right images; and
apply an edge enhancement and depth-based light technique to the filtered images.

19. The system of claim 13, wherein the display of microscope system comprises a left ocular and a right ocular, and
wherein the image generator and controller are configured to display the stereoscopic right image and the right image in the left ocular and the right ocular, respectively.

20. The system of claim 13, wherein the image generator and controller are configured to display the stereoscopic left image and the right image in one of a heads-up display, a video screen, and video goggles.

21. The system of claim 13, wherein the image generator and controller are configured to:
display the stereoscopic left image and the right image of the subject from a first perspective;
receive input via a user interface for changing the display of the subject to a second perspective different than the first perspective; and
in response to receipt of the input:
generate another stereoscopic left image and right image of the subject based on the cross-sectional images; and
display the other stereoscopic left image and the right image in the display of the microscope system.

22. The system of claim 13, wherein the image generator and controller are configured to display at least one of the cross-sectional images in the display of the microscope system.

23. The system of claim 22, wherein the user interface comprises a foot pedal controller.

24. The system of claim 13, wherein the plurality of cross-sectional images are a first plurality of cross-sectional images,
wherein the stereoscopic left image and the right image are a stereoscopic first left image and a first right image;
wherein the image generator and controller are configured to:
capture a plurality of cross-sectional images comprises capturing the first plurality of cross-sectional images within a first time period;
capture a second plurality of cross-sectional images of the subject;
generate a stereoscopic second left image and second right image of the subject; and
display the stereoscopic second left image and second right image in the display at a time different than the display of the stereoscopic first left image and the first right image.

25. A computer program product comprising a computer readable storage medium having program instructions embodied therewith, the program instructions readable by a computing device to cause the computing device to:
capture, by the computing device, a plurality of cross-sectional images of a subject;
generate, by the computing device, a stereoscopic left image and right image of the subject based on the cross-sectional images; and
display, by the computing device, the stereoscopic left image and the right image in a display of a microscope system.

26. The computer program product of claim 25, wherein the subject comprises an eye.

27. The computer program product of claim 25, wherein the subject is a retina of an eye.

28. The computer program product of claim 25, wherein the program instructions cause the computing device to capture a plurality of B-scan images of the subject.

29. The computer program product of claim 25, wherein the program instructions cause the computing device to use an optical coherence tomography (OCT) technique for capturing the cross-sectional images.

30. The computer program product of claim 25, wherein the program instructions cause the computing device to:
filter the left and right images; and
apply an edge enhancement and depth-based light technique to the filtered images.

31. The computer program product of claim 25, wherein the display of microscope system comprises a left ocular and a right ocular, and
wherein the program instructions cause the computing device to display the stereoscopic right image and the right image in the left ocular and the right ocular, respectively.

32. The computer program product of claim 25, wherein the program instructions cause the computing device to display the stereoscopic left image and the right image in one of a heads-up display, a video screen, and video goggles.

33. The computer program product of claim 25, wherein the program instructions cause the computing device to:
display the stereoscopic left image and the right image of the subject from a first perspective;
receive input via a user interface for changing the display of the subject to a second perspective different than the first perspective; and
in response to receipt of the input:
generate another stereoscopic left image and right image of the subject based on the cross-sectional images; and
display the other stereoscopic left image and the right image in the display of the microscope system.

34. The computer program product of claim 25, wherein the program instructions cause the computing device to display at least one of the cross-sectional images in the display of the microscope system.

35. The computer program product of claim 34, wherein the user interface comprises a foot pedal controller.

36. The computer program product of claim 25, wherein the plurality of cross-sectional images are a first plurality of cross-section images,
wherein the stereoscopic left image and the right image are a stereoscopic first left image and a first right image;
wherein the program instructions cause the computing device to:
capture the first plurality of cross-sectional images within a first time period;
capture a second plurality of cross-sectional images of the subject;
generate a stereoscopic second left image and second right image of the subject; and
display the stereoscopic second left image and second right image in the display at a time different than the display of the stereoscopic first left image and the first right image.
